(19) United States
(12) Patent Application Publication
(10) Pub. No.: US 2010/0121219 A1
(43) Pub. Date: May 13, 2010

(54) Mouth Cell Collection Device with Mechanically Rinsible Scoop

(76) Inventors: Edward R.B. McCabe, Pacific Palisades, CA (US); Yao Hua Zhang, Los Angeles, CA (US); Bing Ling Huang, Los Angeles, CA (US); Nicole K. Henderson-MacLennan, Malibu, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/614,290

(22) Filed: Nov. 6, 2009

Related U.S. Application Data
(60) Provisional application No. 61/112,650, filed on Nov. 7, 2008.

(51) Int. Cl. A61B 10/02

(52) U.S. Cl. 600/570; 600/562

(57) ABSTRACT

The present invention provides a collection device for obtaining a sample from within the mouth of a subject. The collection device comprises a collection wand, having a hollow shaft and a scoop attached to the end of the shaft, and optionally, a bulb, a pipetting device, and a collection tube. Also provided are methods of using the collection device to obtain a sample from the mouth of a subject.

### CROSS-REFERENCES TO RELATED APPLICATIONS

This application claims the benefit of priority to U.S. Provisional Application No. 61/112,650, filed on Nov. 7, 2008, which is herein incorporated by reference.

### BACKGROUND OF THE INVENTION

Non-invasive and painless buccal cell isolation methods involving saliva, mouthwash, treated filter paper, cytobrush and foam or cotton-tip swabs are available for collecting buccal cells from humans for diagnostic analyses (1,3,4). These methods, however, are extremely difficult, if not impossible, to adapt for the small size of, for example, the newborn mouse. For example, the filter paper on the Bode Buccal DNA collector is about the width of a neonatal mouse head (1). The Oragene™ saliva collection device requires 1 ml of saliva sample to extract sufficient DNA for processing, or approximately the total weight of the 1 g neonatal mouse (DNA Genotek Inc.). Traditional cotton swabs are much too large to collect buccal cells from a day of life (DOL) 1 mouse and would have the potential to suffocate the mouse if forced into the mouth. Although comparison of cytobrush, mouthwash and treated card for obtaining human buccal cells found that the cytobrush was the best method for human sampling (3), these approaches cannot be adapted directly to mice, especially DOL1 mice. A serrated pipette tip used to collect human cells for RNA isolation is too abrasive for the fragile DOL1 mouse cheek (4). Mouse pups require methods of handling that minimize pain and stress, since they are very fragile and can die easily during execution of the experimental protocol.

Protocols have been developed to address minimally invasive and painless mouse buccal cell sampling. One method for sampling and extraction of mouse DNA used a cotton swab adapted for adult, but not neonatal, mice (2). A common toothpick, though more appropriate in size, would lack a reservoir for collecting adequate buccal cells from the newborn mouse for subsequent analyses. Kits involving a buccal brush or swab (Epicentre), mouse saliva (10 μl; Sigma), and buccal cells applied on a card with a swab (Whatman) are examples of commercial products available for adult mice. However, the youngest mouse described in the protocols for these products was 1 month old.

To date, known, standardized, relatively non-invasive methods for buccal cell sampling from newborn mice are not available from commercial sources or in the published literature. Therefore, there is a need for a less invasive, non-mutilating approach to obtain cells from animals, such as neonatal mice, for experimental procedures that require an oral sample, such as buccal cell sampling, for example for DNA extraction and genotyping.

### BRIEF SUMMARY OF THE INVENTION

In accordance with one aspect of the present invention, there is provided a sample collection device, comprising a collection wand having a shaft, the shaft having a top end and a bottom end, and wherein the shaft has a hollow interior, and a scoop attached to the bottom end of the shaft, wherein the scoop is configured for collecting a sample of cells from within a mouth of a subject and wherein the scoop is sized and shaped to fit within the mouth cavity of the subject without causing significant discomfort to the subject.

In accordance with another aspect of the present invention, there is provided a kit comprising (a) a collection device according to the present invention and (b) instructions for the use thereof.

In accordance with yet another aspect of the present invention, there is provided a method for collecting a sample of cells from a subject, comprising (a) maintaining the mouth of the subject open; and (b) collecting the sample of cells from the mouth of the subject using a collection device according to the present invention.

### BRIEF DESCRIPTION OF THE DRAWINGS

FIG. 1 depicts an example of a front view and a side view of a sample collection wand of the present invention that is suitable for use in small animals, and a sample collection tube suitable for the sample collection wand.

FIG. 2 depicts an example of a front view and a side view of a sample collection wand of the present invention that is suitable for use in humans, and depicts an example of a sample collection device of the present invention including a collection wand having a transfer pipette bulb and a suitable sample collection tube in a closed condition. FIG. 2 also depicts the shape of the scoop portion of the collection wand of the present invention, and depicts the cell collection material that may be placed in the scoop portion.

FIG. 3. Comparison of mouse age versus total cell count of collected buccal cells. Total cell count reflects the total number of cells present in cell pellets resuspended in 20 μl. Dol 1, n=20 from 9 litters. Dol 7, n=15 from 7 litters. Dol 21, n=12 from 6 litters.

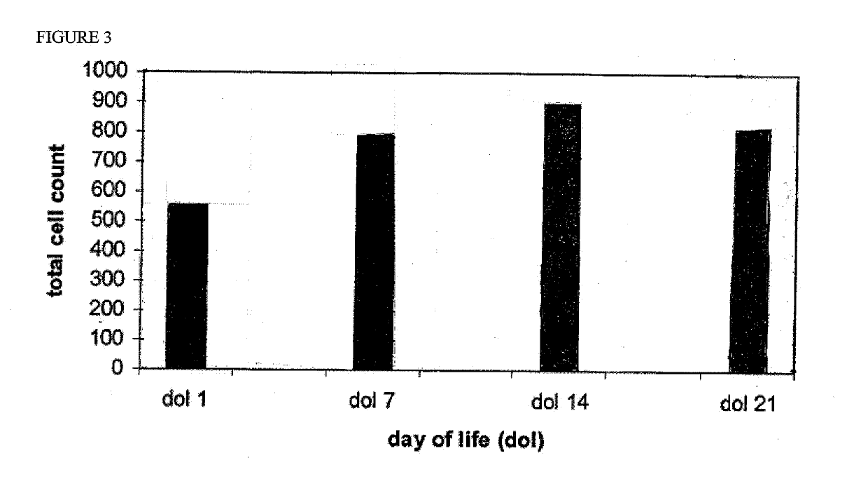

FIG. 4. DMD gene amplification from buccal cell DNA. Lanes 1-4, DMD gene, 10 μl/well. Lane 5, PCR blank. Lane 6, 100 by ladder.

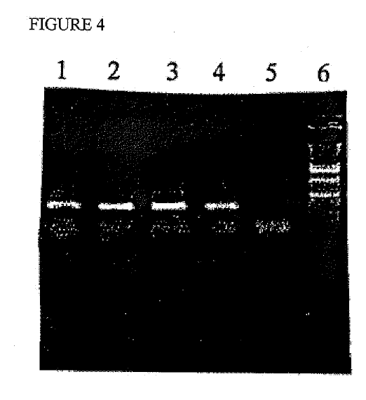

### DETAILED DESCRIPTION OF THE INVENTION

As will be described in more detail below, the present invention is directed to a sample collection device comprising a collection wand sized and configured for sample collection from within the mouth of a subject without causing significant discomfort to the subject. The sample collection device optionally includes a bulb or a pipetting device that attaches to the end of the collection wand and/or a collection tube. The present invention further pertains to a method of use of the collection device to obtain an oral sample from a subject.

Discomfort in a subject, in particular a laboratory animal, is observed by, for example, struggle and/or movements back and forth and/or vocalizations and avoidance by the subject, which is typically observed when the subject is in pain or under stress. Such discomfort is observed in known mutilating procedures such as total or partial amputation of the laboratory animal's toe, ear or tail. Similar or corresponding discomfort reactions in response to pain or stress are observed in both neonatal and non-neonatal subjects, and would be readily recognizable by the skilled worker. The methods and devices of the present application permit an oral sample to be obtained from the subject without causing discomfort or without causing significant discomfort. A worker skilled in the art, having experience working with subjects such as laboratory animals, would readily appreciate what is meant by “significant discomfort.” In a subject experiencing no discomfort there would be no observation of the discomfort reactions described above. In a subject experiencing mild discomfort, but not significant discomfort, some discomfort reactions would be observed but the discomfort should be tolerable to the subject.

The terms “oral sample”, as used herein, includes samples obtained from within the mouth of a subject that contains one or more of: saliva, sputum, microorganisms (including viral, bacterial, fungal, protozoan, parasitic, single-celled, and/or multi-cellular organisms) present in the mouth, cells, buccal mucosa and/or buccal cells.

The term “subject”, as used herein, refers to a mammal, for example, a human or a non-human mammal, including primates (e.g., macaque, pan troglodyte, pongo), a domesticated mammal (e.g., felines, canines), an agricultural mammal (e.g., bovine, ovine, porcine, equine) and a laboratory mammal or rodent (e.g., rattus, murine, lagomorpha, hamster).

For descriptive purposes herein, two embodiments of a sample collection device of the invention will be described with reference to the figures. One of the illustrated embodiments may be particularly suited for collecting an oral sample from the mouth of a neonatal rodent, such as a neonatal mouse. Another of the illustrated embodiments may be particularly suited for collecting an oral sample from the mouth of a neonatal human subject. However, the illustrated embodiments would be readily adaptable for use in other subjects, such as small animals, including neonatal and non-neonatal laboratory animals, such as mice, rats, hamsters and rabbits, and larger animals, including neonatal and non-neonatal laboratory animals, such as horses. Thus, according to these embodiments, the scoop portion at the working end of the collection wand can be passed into the mouth of a mouse, human, or other species and directed towards a part of the mouth such as the inner cheek or buccal pouch. Once at a desired location the collection wand can be manipulated to facilitate sample collection.

Reference to the figures is not intended to limit the scope of the invention.

### Collection Device

### Collection Wand

The collection device comprises a collection wand having a shaft and a scoop attached to the shaft. The shape and length of the shaft and scoop with vary according to the intended use, needs, preferences, and/or choice of construction materials.

The shaft may be conical or cylindrical in shape. The shaft has a top end and a bottom end. In some embodiments, the opening of the top end may be wider in diameter than the opening of the bottom end. In other embodiments, the opening of the top end and the opening of the bottom end may be about the same size. The shaft has a hollow interior. In some embodiments, the shaft remains open at both the top end and the bottom end. In other embodiments, the shaft remains open at the bottom end, but has a bulb or pipetting device attached to the top end. The dimensions of the shaft are determined based on the average size of the mouth of the subject (species and age) from which the sample is to be collected. For some subjects, for example a neonatal mouse, the shaft is less than about 35 mm in length. For other subjects, for example a neonatal human, the shaft is less than about 100 mm in length. For still other subjects, for example a neonatal horse, the shaft is less than about 105 mm in length. In some preferred embodiments, the dimensions of the scoop are about 33 mm in length. In other preferred embodiments, the dimensions of the scoop are about 95 mm in length. In still other preferred embodiments, the dimensions of the scoop are about 100 mm in length. These dimensions are only some examples of the dimensions that are possible for the present invention. One of skill in the art will recognize that the shaft may be made smaller or larger than the dimensions described above in order to accommodate different mouth sizes.

The scoop is attached to the bottom end of the shaft. The scoop has a collection reservoir for the oral sample collected from the mouth of the subject. In preferred embodiments, the scoop has a collection reservoir for bucchal cells scraped from the cheek of the subject. The collection reservoir is concave in shape.

The scoop is sized and shaped to fit within the mouth of the subject without causing significant discomfort to the animal. In some embodiments, the shape of the scoop resembles a crescent moon. In other embodiments, the shape of the scoop resembles a half sphere. The dimensions of the scoop are determined based on the average size of the mouth of the subject (species and age) from which the sample is to be collected. For some subjects, for example a neonatal mouse, the scoop is less than about 2 mm in width. For other subjects, for example a neonatal human, the scoop is less than about 6 mm in width. For still other subjects, for example a neonatal horse, the scoop is less than about 14 mm in width. Similarly, the length of the scoop will vary depending on the subject. For some subjects, for example a neonatal mouse, the scoop is less than about 4 mm in length. For other subjects, for example a neonatal human, the scoop is less than about 6 mm in length. For still other subjects, for example a neonatal horse, the scoop is less than about 45 mm in length. In some preferred embodiments, the dimensions of the scoop are about 1.5 mm in width and about 3 mm in length. In other preferred embodiments, the dimensions of the scoop are about 5 mm in width and about 5.5 mm in length. In still other preferred embodiments, the dimensions of the scoop are about 12 mm in width and about 40 mm in length. These dimensions are only some examples of the dimensions that are possible for the present invention. One of skill in the art will recognize that the scoop may be made smaller or larger than the dimensions described above in order to accommodate different mouth sizes.

The scoop may be made from a plastic material or any flexible material that can retain the shape of a scoop. In some embodiments, the scoop may be made from a thin cardboard-like material coated with a material that enables cells to stick to it. The material could be inherently sticky for cells, or alternatively, could be treated in a process such as the one disclosed in U.S. Pat. No. 6,617,152 (herein incorporated by reference in its entirety), in which the use of a microwave process for treating the culture surface improves cell attachment by incorporating significantly more oxygen into the cell culture surface, rendering it more hydrophilic and increasing surface stability. Alternatively, the scoop may be made from or coated with new dental materials such as polymethylmethacrylate (PMMA). The scoop may be made from a single material or any combination of materials. In preferred embodiments, the scoop is made out of a single layer of plastic, which is more pliable than a device made from two layers of plastic and therefore conveys the advantage of greater pliability, which will facilitate collection of more cells from the mouth. The shaft may be made from the same material as the scoop or alternatively may be made from a different material than the scoop. In preferred embodiments, the shaft is made from a plastic material.

In some embodiments, the scoop may include cell collection material. This cell collection material is an absorbent material that facilitates the collection and retention of cells until the user is ready to release the cells into the collection liquid. The absorbent material may be made from cotton, from a cotton-like material, or any combination thereof. Additionally, the absorbent material may be wrapped in a mesh. This mesh may be made from a plastic material, an elastic material, or any combination thereof. The cell collection material may be added to any size of cell collection device of the present invention, and the size of the cell collection material may be increased or decreased depending on the dimensions of the scoop into which the cell collection material is being placed. The cell collection material provides the advantage of retaining cells until the user is ready to release the cells, and provides the additional advantage of eliminating the retention of contaminating debris due to the mesh surrounding the absorbent material.

In some embodiments, the collection wand may also include a pipetting device attached to the top end of the shaft. In other embodiments, a bulb may be attached to the top end of the shaft. When a pipetting device or bulb is attached to the shaft, it confers on the collection device an automated-rinsing capability. The pipetting device can be utilized, or the bulb can be squeezed and released, in order to draw liquid into and out of the shaft and across the scoop in order to remove cells from the scoop and suspend them in the liquid. Drawing fluid up and down in the shaft will facilitate greater cell removal from the scoop of the cell collection device, which is critical for obtaining the maximum amount of cells from the sample; in contrast, a cell collection device without this automated-rinsing capability must be shaken or vortexed in a tube to remove cells from the scoop, and fewer cells are collected by such a method than is possible by using the present invention. One of skill in the art will recognize that a bulb can be attached to any size of cell collection device of the present invention in order to provide the device with self-rinsing capability.

### Collection Tube

In accordance with one embodiment of the present invention, the collection device includes a collection tube for storing the oral sample. In this embodiment, the collection wand is configured to fit within the collection tube.

In some embodiments, the collection tube is a 1.5 mL microcentrifuge tube. In other embodiments, the collection tube is a 15 mL conical tube. In still other embodiments, the collection tube is a 50 mL conical tube. These tubes are of the type that would be known to the skilled worker.

The collection tube can be manufactured from any suitable material including a polymeric material, such as plastics commonly used in the art for similar sample material collection and/or performance of analyses as will be apparent from reading the present disclosure. Examples of suitable plastics include polyurethane, polystyrene, polyvinyl, polypropylene, polyurethane, etc. Desirably, the sample collection device, including the collection wand and the collection tube, is amenable to sterilization during or after manufacture either as an assembled unit or as separate components.

### Optional Components

The cell collection device of the present invention can be combined with other components. For example, the device can be included as a standard part of an oral surgical, dentistry, or forensic procedure to collect cells to look for problems such as cancer or poisoning. For example, using the cell collection device of the present invention, a scientist can perform a quick colorimetric assay in the tube holding the freshly collected cells to determine if a specific poison (e.g. arsenic) is present.

### Collection Method

In accordance with another aspect of the present invention there is provided a method of obtaining an oral sample from a subject, such as a laboratory animal or a human subject, for example a neonatal laboratory animal or a neonatal human.

The method generally comprises the steps of opening the mouth of the subject to be sampled, inserting the scoop portion of the collection wand into the subject's mouth, with the concave portion of the scoop facing vertically up, gently pressing the collection wand on the mucosal surface, moving or rotating the scoop portion of the collection wand within the subject's mouth to obtain the oral sample, and extracting the scoop portion of the collection wand together with the obtained sample from the subject's mouth. Such movement or rotation can additionally include the step of rubbing or scraping a surface within the mouth of the animal, such as, but not limited to, the inner cheek, tongue, roof of the mouth, sublingual area and gums.

In one specific embodiment of the present invention, the scoop of the collection wand is used to collect an oral sample from the inner cheek (such as the cheek pouches/buccal cavity) of a subject. In a preferred embodiment, the scoop portion is used to collect buccal mucosa containing buccal cells. It will be clear that in addition to, or alternatively, the oral sample may be obtained from other areas of the mouth of the subject, such as the tongue, gums, roof of the mouth, sublingual area, palate and/or the like.

In some embodiments, the cell collection method may be practiced using a collection wand further comprising a cell collection material in the scoop portion. This method generally comprises the steps of opening the mouth of the subject to be sampled, inserting the scoop portion of the collection wand into the subject's mouth, with the concave portion of the scoop facing vertically up, gently pressing the collection wand on the mucosal surface, moving or rotating the scoop portion of the collection wand containing the cell collection material within the subject's mouth to obtain the oral sample, and extracting the scoop portion of the collection wand together with the obtained sample from the subject's mouth. The cell collection material facilitates the collection of the sample from the subject's mouth and retains the collected cells until the user is ready to release the cells into a collection liquid.

In some embodiments, the cell collection method may be practiced by rinsing the cells from the scoop portion of the collection wand. This method generally comprises the steps of opening the mouth of the subject to be sampled, inserting the scoop portion of the collection wand into the subject's mouth, with the concave portion of the scoop facing vertically up, gently pressing the collection wand on the mucosal surface, moving or rotating the scoop portion of the collection wand within the subject's mouth to obtain the oral sample, extracting the scoop portion of the collection wand together with the obtained sample from the subject's mouth, and placing the scoop portion in a liquid. The operation end of the collection wand is then attached to a pipetting device so that fluid can be drawn up and down in the shaft of the collection wand, thus facilitating greater cell removal from the scoop portion and suspending the cells in liquid. Alternatively, the operation end of the collection wand may then be attached to a bulb, or a bulb may be pre-attached to the operation end of the collection wand, and the bulb then squeezed and released in order to draw liquid into and out of the shaft of the collection wand and across the scoop portion, in order to remove the cells from the scoop portion and suspend them in liquid.

In one of the specific embodiments of the present invention, a bulb is attached to the operation end of a collection wand suitable for use in a human subject. However, a bulb can be attached to the operation end of any size of collection wand of the present invention. For example, a bulb can be attached to the operation end of a collection wand that is suitable for use in rodent subjects, for example in a neonatal laboratory rodent subject.

A sample collection device according to the invention can be used for collecting oral samples that can be analyzed using, for example, PCR analysis, other methods of nucleic acid-based diagnostics, immunochemistry, biochemical analysis, microbial culture, mass spectrometry, and biosensor-based detection etc. Embodiments of the invention, however, are not limited to these examples. One of ordinary skill in the art will appreciate other types of analyses which may be performed on samples collected by the sample collection device disclosed herein.

In the case in which the method of the present application is used to isolate nucleic acids from the oral sample, the method further comprises the step of isolating nucleic acid contained within the oral sample.

The term “nucleic acid”, as used herein, refers to a chain of nucleotides and includes deoxyribonucleic acid (DNA) or ribonucleic acid (RNA), typically found in chromosomes, chromatin, mitochondria, ribosomes, bacteria or viruses. In one example, the nucleic acid isolated by the method of the present invention is DNA or RNA. In a specific example, the isolated nucleic acid is DNA.

It will be clear that although the isolation of nucleic acids from an oral sample is exemplified, the present invention is suitable for the isolation and/or analysis of other compounds present within the oral sample. Non-limiting examples of such other compounds include peptides, proteins, hormones, chemicals and the like.

The present methods are also suited to generating cell lines from cells within the oral sample collected.

Methods of the invention are conveniently practiced by providing the collection device, including the collection wand and, optionally, one or more of the cell collection material, bulb, and collection tube in the form of a kit. Such a kit preferably contains appropriate instructions for use. Additionally, in the case in which the oral sample is to be used in a downstream PCR, the kit can also include primers and/or one a positive control useful in the detection of a target gene, DNA, RNA.

In one example, a kit comprises (a) a collection wand and (b) instructions for the use thereof. In another example, a kit comprises (a) a collection wand, (b) a bulb, and (c) instructions for the use thereof. In another example, a kit comprises (a) a collection wand, (b) a bulb, (c) a collection tube, and (d) instructions for the use thereof. In yet another example, a kit comprises (a) a collection wand, (b) cell collection material, (c) a bulb, (d) a collection tube and (e) instructions for the use thereof.

To gain a better understanding of the invention described herein, the following examples are set forth. It should be understood that these examples are for illustrative purposes only. Therefore, they should not limit the scope of this invention in any way.

### Examples

### Example 1

This example describes the design and use of a mouse mouth collection device according to a specific embodiment of the present invention. This device can be used without causing discomfort, or without causing significant discomfort, to the mouse. Veterinarians and researchers look for the least harmful and least invasive method for procedures on an animal.

Rodent Mouth Cell Collection Device with Mechanically Rinsible Scoop

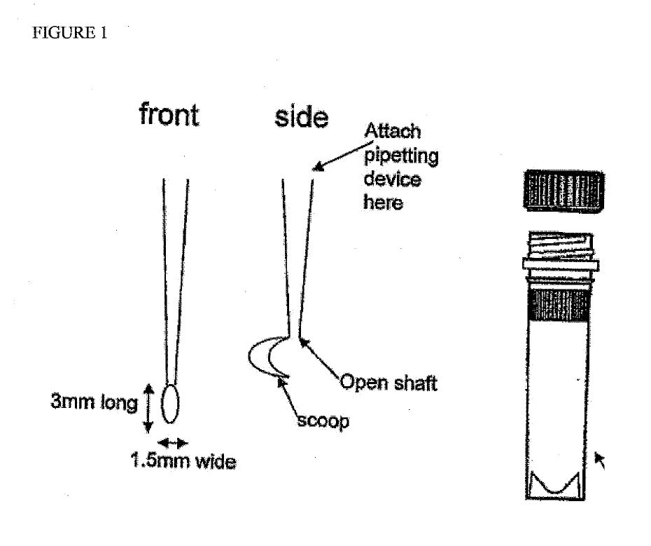

FIG. 1 depicts a mouth cell collection device with a mechanically rinsible scoop that is suitable for use in a rodent, for example a neonatal mouse. The rodent mouth cell collection device is a sterile, disposable mucosal collection device designed to collect the optimum specimen with little or no discomfort to the mouse subject. The device may be packaged in a sterile condition or in packaging that could be sterilized by the user, and removed by the stem of the device.

The collection wand of the collection device comprises a hollow shaft, and for the purposes of this example the shaft is 33 mm long. The collection wand has a wide end and a narrow end, and the narrow end of the collection wand is modified to have a miniature concave scoop, while the holes at both ends of the shaft remain open. Because both ends of the shaft remain open, fluid may be drawn up and down in the shaft, which facilitates greater cell removal from the scooped-shaped portion of the collection wand. The collection wand is made of one layer of plastic, giving it the advantage of being more pliable, which facilitates collection of more cells from the rodent mouth. However, the collection wand may be made out of any flexible material that can retain the shape of the scoop. The scoop is shaped like a crescent moon, and has a concave reservoir for cell collection. For the purposes of this example, the size of the scoop is 1.5 mm wide by 3 mm long. At the wide end of the collection wand, a pipetting device may be attached that can be used to rinse off the scoop to increase cell yield. The cell collection device also includes a collection tube, which for the purposes of this example is a 1.5 mL microcentrifuge tube.

### Mouth Cell Collection Process

In this example, buccal cells are scraped from the cheek of a mouse subject. The rodent mouth cell collection device is inserted into the mouse's mouth, always with the concave portion of the scoop facing vertically up, so that the cells do not fall out. The device is gently pressed on the mucosal surface and moved outwards 3-5 mm. Five repetitions of this motion are carried out on the left and right cheek pouches with a rinse in between in a microcentrifuge tube containing the liquid (e.g., 1 mL of 0.9% saline). Each cheek is supported from the outside by a finger during scraping. After scraping both cheeks, the cells are then deposited into the same microcentrifuge tube containing the liquid (e.g., 1 mL of 0.9% saline), by attaching to and rinsing with a pipetting device. The cell suspension is centrifuged (e.g., at 14,000 rpm or 15,800 g for 10 sec.) and the supernatant is discarded. Cells can then be used for culturing or to extract DNA, e.g. for genotyping, or RNA and protein, e.g. for expression studies. Notably, by using this method, cells—not saliva—are collected.

### Example 2

This example describes the design and use of a human mouth collection device according to a specific embodiment of the present invention. This device can be used without causing discomfort, or without causing significant discomfort, to the human subject. Researchers look for the least harmful, most efficient, and least invasive method for procedures on a human subject, especially for use in human babies and children.

Human Mouth Cell Collection Device with Built-In Rinsible Scoop

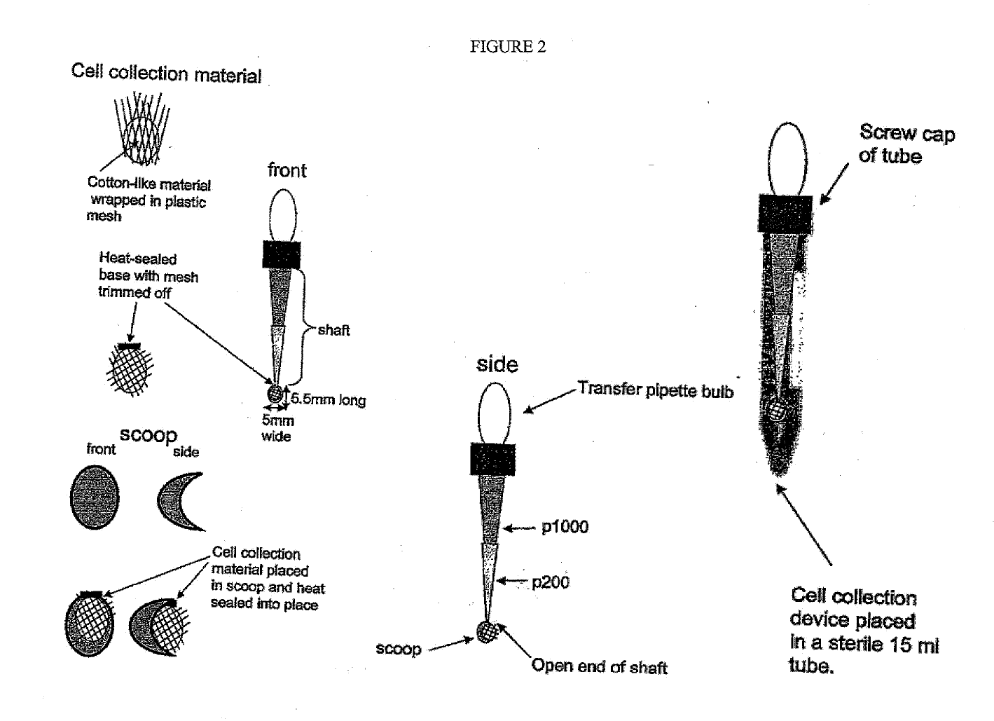

FIG. 2 depicts a mouth cell collection device with a mechanically rinsible scoop that is suitable for use in a human, for example a neonatal human. The human mouth cell collection device is a sterile, disposable mucosal collection device designed to collect the optimum specimen with little or no discomfort to the human subject. The device may be packaged in a sterile condition or in packaging that could be sterilized by the user, and removed by the bulb or lid of the device.

The collection wand of the collection device comprises a hollow shaft, and for the purposes of this example the shaft is 95 mm long. The collection wand has a wide end and a narrow end, and the narrow end of the collection wand is modified to have a miniature concave scoop, while the holes at both ends of the shaft remain open. Because both ends of the shaft remain open, fluid may be drawn up and down in the shaft, which facilitates greater cell removal from the scooped-shaped portion of the collection wand. The collection wand is made of one layer of plastic, giving it the advantage of being more pliable, which facilitates collection of more cells from the rodent mouth. However, the collection wand may be made out of any flexible material that can retain the shape of the scoop. The scoop is shaped like a half sphere, and has a concave reservoir for cell collection. For the purposes of this example, the size of the scoop is 5 mm wide by 5.5 mm long. At the wide end of the collection wand, a transfer pipette bulb is attached that can be used to rinse off the scoop to increase cell yield. The cell collection device also includes a collection tube, which for the purposes of this example is a 15 mL conical tube.

### Mouth Cell Collection Process

In this example, buccal cells are scraped from the cheek of a mouse subject. The rodent mouth cell collection device is inserted into the mouse's mouth, always with the concave portion of the scoop facing vertically up, so that the cells do not fall out. The device is gently pressed on the mucosal surface and moved outwards approximately 5 mm. One repetition of this motion is carried out on the left and right cheek pouches with a rinse in between in a 15 mL conical tube containing the liquid (e.g., 1 mL of 0.9% saline). The cheek is supported from the outside by a finger during scraping. After scraping both cheeks, the cells are then deposited into the same 15 mL conical tube containing the liquid (e.g., 1 mL of 0.9% saline), by attaching to and rinsing with a pipetting device. The cell suspension is centrifuged (e.g., at 14,000 rpm or 15,800 g for 10 sec.) and the supernatant is discarded. Cells can then be used for culturing or to extract DNA, e.g. for genotyping, or RNA and protein, e.g. for expression studies. Notably, by using this method, cells—not saliva—are collected.

### REFERENCES

1. Burger, M. F., E. Y. Song and J. W. Schumm. 2005. Buccal DNA samples for DNA typing: new collection and processing methods. Biotechniques 39:257-261.
2. Meldgaard, M., P. J. Bollen and B. Finsen. 2004. Non-invasive method for sampling and extraction of mouse DNA for PCR. Lab Anim 38:413-417.

3. Mulot, C., I. Stucker, J. Clavel, P. Beaune and M. A. Loriot. 2005. Collection of human genomic DNA from buccal cells for genetics studies: comparison between cytobrush, mouthwash, and treated card. J Biomed Biotechnol 2005:291-296.

4. Spira, A., J. Beane, F. Schembri, G. Liu, C. Ding, S. Gilman, X. Yang, C. Cantor and J. S. Brody. 2004. Noninvasive method for obtaining RNA from buccal mucosa epithelial cells for gene expression profiling. Biotechniques 36:484-487.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

What is claimed is:

1. A sample collection device, comprising:
a collection wand having a shaft, the shaft having a top end and a bottom end, and wherein the shaft has a hollow interior, and a scoop attached to the bottom end of the shaft, wherein the scoop is configured for collecting a sample of cells from within a mouth of a subject and wherein the scoop is sized and shaped to fit within the mouth cavity of the subject without causing significant discomfort to the subject.

2. The device of claim 1, further comprising a bulb attached to the top end of the shaft.

3. The device of claim 1, further comprising a pipetting device attached to the top end of the shaft.

4. The device of any of claim 1, wherein the scoop has a concave shape.

5. The device of claim 4, wherein the concave shape is a crescent.

6. The device of claim 4, wherein the concave shape is a half-sphere.

7. The device of claim 1, wherein the scoop further comprises an absorbent material capable of retaining the sample of cells.

8. The device of claim 7, wherein the absorbent material is made from a cotton material, a cotton-like material, or any combination thereof.

9. The device of claim 7, wherein the absorbent material is wrapped in a mesh.

10. The device of claim 9, wherein the mesh is made from a plastic material, an elastic material, or any combination thereof.

11. The device of claim 1, wherein the scoop is from about 1 mm wide to about 15 mm wide.

12. The device of claim 1, wherein the scoop is from about 3 mm long to about 50 mm long.

13. The device of claim 1, wherein the scoop is about 1.5 mm wide and about 3 mm long.

14. The device of claim 1, wherein the scoop is about 5 mm wide and about 5.5 mm long.

15. The device of claim 1, wherein the scoop is about 12 mm wide and about 40 mm long.

16. The device of claim 1, wherein the shaft is from about 30 mm long to about 150 mm long.

17. The device of claim 13, wherein the shaft is about 33 mm long.

18. The device of claim 14, wherein the shaft is about 95 mm long.

19. The device of claim 15, wherein the shaft is about 100 mm long.

20. The device of claim 1, wherein the scoop is made from a plastic material, a flexible material, or any combination thereof.

21. The device of claim 20, wherein the scoop is made from a single layer of a plastic material, a flexible material, or any combination thereof.

22. The device of claim 1, wherein the scoop is coated with a material that facilitates the sticking of the sample of cells to the scoop.

23. The device of claim 1, further comprising a collection tube.

24. The device of claim 23, wherein the collection tube includes a removable cap.

25. The device of claim 23, wherein the collection tube is a microcentrifuge tube.

26. The device of claim 23, wherein the collection tube is a conical tube.

27. A kit comprising:
(a) a collection device according to claim 1; and
(b) instructions for the use thereof.

28. A method for collecting a sample of cells from a subject, comprising:
(a) maintaining the mouth of the subject open; and
(b) collecting the sample of cells from the mouth of the subject using a collection device according to claim 1.

29. The method of claim 28, wherein step (b) comprises inserting the scoop of the collection device into the mouth of the subject, pressing the device against a mucosal surface of the mouth, and scraping the mucosal surface with the scoop.

30. The method of claim 29, additionally comprising the step of (c) drawing a liquid into and out of the shaft and across the scoop, thereby removing the sample of cells from the scoop and suspending the sample of cells in the liquid.

31. The method of claim 30, additionally comprising the step of (d) isolating nucleic acid or protein contained with the sample of cells.

32. The method of claim 28, wherein the subject is a human.

33. The method of claim 28, wherein the subject is a neonate.
